(19) United States
(12) Patent Application Publication
(10) Pub. No.: US 2020/0015506 A1
(43) Pub. Date: Jan. 16, 2020

(54) PARTICULATE HEALTH FOOD

(71) Applicant: CHIBA MACHIKO COOKING STUDIO YUGEN KAISHA, Tokyo (JP)

(72) Inventor: Machiko CHIBA, Tokyo (JP)

(21) Appl. No.: 16/036,194

(22) Filed: Jul. 16, 2018

(51) Int. Cl. A23L 31/00; A23L 33/105; A61K 36/07; A61K 36/82

(52) U.S. Cl. CPC A23L 31/00; A23L 33/105; A61K 36/07; A61K 36/82; A23V 2300/26; A23V 2200/30; A23V 2250/214; A23V 2200/044; A23V 2002/00

(57) ABSTRACT

A particulate health food includes 100 parts by mass of particulate powdered wood ear mushrooms having a particle diameter of 125 to 250 μm, and 15 to 35 parts by mass of matcha having a particle diameter of 1 to 100 μm. A matcha particle of a relatively large particle size and a matcha particle of a relatively small particle size are closely loaded onto surfaces of a powdered wood ear mushroom such that the small matcha particle enters a gap between the large matcha particles. Matcha particles having two different particle diameters and having a brighter color than wood ear mushrooms firmly adhere to surfaces of the powdered wood ear mushroom so that the matcha particles are integrated and become hard to separate from each other even under vibration, and the powdered wood ear mushroom is made more palatable.

### TECHNICAL FIELD

The present invention relates to a particulate health food that includes powdered dry wood ear mushrooms as a main component.

### BACKGROUND ART

A wood ear mushroom is a fungus (fruiting body) belonging to heterobasidiomycetes of Basidiomycota (in the order Auriculariales or Tremellales), well known as an edible mushroom often growing on decaying logs of many different kinds of coniferous or broadleaf trees including elders and oaks, among others.

These wood ear mushrooms constitute a low-calorie food containing well-balanced nutrients needed for physical health, such as protein, calcium, iron, fiber, and vitamin D. This is a distinct feature of wood ear mushrooms not seen in other general foods.

In particular, percentages of dietary fibers and Vitamin D contained in a wood ear mushroom are so high that they come in first or second place in all the food groups listed in the Standard Tables of Food Composition in Japan. Thus, wood ear mushrooms, when used as a food for diet or other health purposes, provide a very helpful health or diet food in the aging society. For example, such food is far more unlikely to cause physical problems than diet foods made from konjac.

These edible wood ear mushrooms (both black and white ones) are commercially available in the form of a highly preservable dried product. Before cooking, the dried wood ear mushrooms are soaked in water to be softened with absorbed water.

When swollen from the original dried material to be cooked, black wood ear mushrooms become crunchy and white ones become gelatinous. Both black and white wood ear mushrooms are often added to noodles, ready-made dishes, and other various meals because such mushrooms have light flavor. However, these wood ear mushrooms are seldom served as an independent food because they originally lack a strong, distinctive taste.

Through previous patent applications, the inventor of the present application disclosed a health food produced by adding a binding agent to particulates of dried wood ear mushrooms, mixing them, and compressing the mixture into a tablet (Patent Document 1), and also disclosed a health food produced by loading particulates of dried wood ear mushrooms into a water-soluble capsule (Patent Document 2).

Meanwhile, known health supplements that have a low calorie content and may serve dieting purposes include a supplement containing particulates of dried wood ear mushrooms and dried hijiki seaweed, kelp root, hijiki seaweed, wakame seaweed, mozuku seaweed, black sesame, and black rice that have been powdered (Patent Document 3).

### PRIOR ART REFERENCES

### Patent Documents

Patent Document 1: Japanese Patent No. 2872635

Patent Document 2: Japanese Unexamined Patent Application Publication No. H11-113523

Patent Document 3: Japanese Unexamined Patent Application Publication No. 2002-300860

### SUMMARY OF THE INVENTION

### Technical Problem

However, particulates obtained by crushing a product of the above-mentioned known dried wood ear mushrooms are made up of particles that are dry, hard, and angular, and thus are difficult to be homogeneously mixed with particulates of other components. Even though these particulates can be mixed homogeneously for the time being, a slight vibration will often cause the particulates to separate into layers according to particle diameter. Therefore, particulates of different components have to be homogeneously mixed immediately before, or at some appropriate time before, the particulates having uniformly distributed components are transferred to a container, divided into small portions, and so on.

In addition, particulate dried wood ear mushrooms are dark brown in color, and thus may need coloring to a certain different color so as to be more palatable as a food. However, the particulates need a large amount of colorant because their surfaces are so hard and angular that it is difficult for the colorant to adhere to and cover the surfaces efficiently, which means the colorant is added with low efficiency.

When mixed with powdered dry wood ear mushrooms, the added components described in Patent Document 3 above do not increase any colorability, failing to change the impression of wood ear mushrooms given by color. Even when the added components serve as a colorant, these components cannot change color or flavor to a sufficient extent unless the components are added in an amount equal to or greater than the amount of wood ear mushrooms.

As seen above, it is conventionally not easy to color dried wood ear mushrooms with a relatively small amount of colorant so as to increase the efficiency of adding a colorant and to color the mushrooms brighter so as to make them more palatable.

In addition, as described above, it is conventionally not easy to keep a homogeneous mixture of particulates until loaded into a capsule because the mixture of particulates is difficult to fit into other components and is apt to separate by particle size in the particle-size distribution when a vibration is given.

Therefore, an object of the present invention is to solve the above-described problems by providing a particulate health food or a capsuled particulate health food that includes powdered dry wood ear mushrooms as a main component, where the particulate health food includes a particulate composition which is sufficiently colored with a coloring component that has a bright color for making the wood ear mushrooms more palatable and that can be added with higher efficiency, and which keeps a homogenous mixture in the form of particulate composition of the wood ear mushrooms and the coloring component to be prevented from separating into layers when the mixture is vibrated before or after loaded into a capsule.

### Solution to Problem

To solve the above-described problems, the present invention provides a particulate health food including a particulate composition that includes wood ear mushrooms as a main component, the wood ear mushrooms comprising powdered particulate dry wood ear mushrooms having a particle diameter of 125 to 250 μm, and wherein the particulate composition further includes, based on 100 parts by mass of the wood ear mushrooms, 15 to 35 parts by mass of matcha (powdered green tea) having a particle diameter of 1 to 100 μm.

The particulate health food as configured above according to the present invention is a versatile health food that can be added to general foods without causing a feeling of strangeness. With the powdered particulate dry wood ear mushrooms having a particle diameter of 125 to 250 μm included as a main component, the particulate health food is made up of a homogeneous mixture forming a composition of 100 parts by mass of the powdered particulate dry wood ear mushrooms having such predetermined particle diameters and a predetermined amount of, that is, 15 to 35 parts by mass of, the matcha having a particle diameter of 1 to 100 μm, which is smaller than the powdered particulate wood ear mushrooms.

In the particulate composition, matcha is efficiently added and adheres to the powdered wood ear mushrooms, where the matcha is smaller in particle diameter, relatively soft, and brighter in color than the powdered wood ear mushrooms, which are made up of dry, hard, and angular particles. Thus, it is made possible to color the powdered wood ear mushrooms to a matcha color so as to make the mushrooms more palatable by adding a small amount of matcha, and to provide a homogeneous particulate health food containing a mixture that is unlikely to separate when any vibration is given, because the matcha adheres to and covers surfaces of the particles of wood ear mushrooms.

In particular, such effect is excellently exerted by the matcha having a particle-size distribution in which peaks appear at 3 to 10 μm and 20 to 40 μm.

In general, it is ensured that the aforementioned desirable results are obtained by using the matcha of 1 to 100 μm in particle diameter, where the particle-size distribution of the matcha shows a peak at 3 to 10 μm corresponding to a finely crushed thin portion of raw green tea leaves and a peak at 20 to 40 μm corresponding to a crushed thick or vein portion of raw green tea leaves.

During a process of forming the above-described composite particles, small and large matcha particles in diameter are attached onto an angular particle of wood ear mushroom having a relatively large particle diameter, with the small particles entering a gap between the large particles. Hence, matcha particles are closely loaded onto a wood ear mushroom particle with a smaller void fraction, with the result that the matcha particles firmly adhere to the wood ear mushroom particles to be integrated with the mushroom particles.

In addition, in cases where the aforementioned particulate health food is a granular particulate composition, the particulate health food is reliably prevented from separating by particle size in the mixture, because the health food can remain more stably while relatively soft and small-diameter matcha particles are adhering to powdered wood ear mushrooms.

Furthermore, the particulate health food made up of a stable, hard-to-separate mixture as described above can be contained in a capsule ingestible with a liquid to be formed into a capsuled particulate health food. When contained in a capsule, the mixture is still hard to separate into matcha particles and powdered wood ear mushroom particles, which are efficiently colored in a matcha color so as to be more palatable.

### Effects of the Invention

According to the present invention, the particulate health food contains 100 parts by mass of powdered wood ear mushrooms having a predetermined particle diameter and a predetermined amount of, that is, 15 to 35 parts by mass of, matcha having a particle diameter of 1 to 100 μm, which is smaller than the powdered wood ear mushrooms. Therefore, the present invention is advantageous in that there is provided a particulate health food or a capsuled particulate health food that includes powdered dry wood ear mushrooms as a main component, where the powdered wood ear mushrooms can be made more palatable by efficiently adding a colorant component of a color brighter than the wood ear mushrooms and the particulate health food does not separate by particle size when the mixture is vibrated before or after loaded into a capsule.

### BRIEF DESCRIPTION OF THE DRAWINGS

FIG. 1 is a schematic diagram illustrating composite particles made of powdered wood ear mushrooms and powdered matcha.

FIG. 2 is a flowchart illustrating a production method according to an embodiment.

### DESCRIPTION OF EMBODIMENTS

A particulate health food according to an embodiment of the present invention includes a particulate composition that includes wood ear mushrooms as a main component, the wood ear mushrooms comprising powdered particulate dry wood ear mushrooms having a particle diameter of 125 to 250 μm, and the particulate composition further including, based on 100 parts by mass of the wood ear mushrooms, 15 to 35 parts by mass of matcha having a particle diameter of 1 to 100 μm.

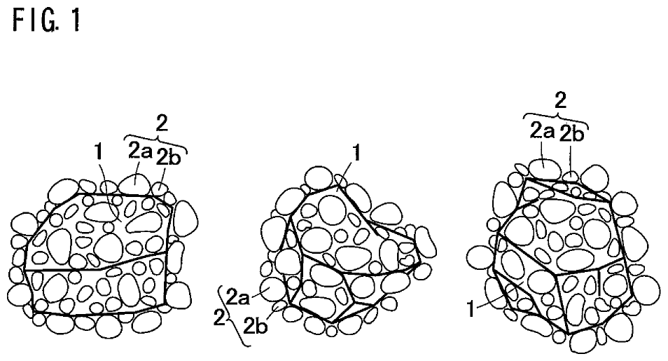

As schematically illustrated in FIG. 1, a powdered wood ear mushroom 1 is dry and angular in particle morphology, has bumps and dips, and is porous and water absorbent. Thus, matcha particles 2, which are relatively soft and have a predetermined smaller particle diameter, are apt to adhere to surfaces of the particle of wood ear mushroom, which means matcha particles and powdered wood ear mushrooms are easy to be homogeneously mixed and to fit into each other.

The matcha particles 2 being 1 to 100 μm in particle diameter have a particle-size distribution showing a peak at 3 to 10 μm corresponding to a finely crushed thin portion of raw green tea leaves and a peak at 20 to 40 μm corresponding to a crushed thick or vein portion of raw green tea leaves.

Thus, a matcha particle 2a of a relatively large particle size and a matcha particle 2b of a relatively small particle size are closely loaded onto surfaces of the powdered wood ear mushroom 1, which is angular, in such a way that the small matcha particle 2b enters a gap between the large matcha particles 2a. As a result, matcha particles 2 having two different particle diameters firmly adhere to surfaces of the powdered wood ear mushroom 1 such that particles as a whole are integrated, and eventually the matcha particles 2 including both components and the powdered wood ear mushroom 1 are hard to separate from each other, even when a vibration is given to these composite particles.

Wood ear mushrooms used for the present invention may be any of known edible wood ear mushrooms, and are not limited to any specific types of mushrooms. Examples of the types of wood ear mushrooms that may be used include Auricularia auricula belonging to the order Auriculariales, its related species such as Auricularia polytricha, Auricularia delicata, and Auricularia mesenterica, or ryusenpai, and Tremella fuciformis belonging to the order Tremellales.

Raw wood ear mushrooms may be dried by using any known method for natural (sun) drying or for forced hot-air drying. In view of preserving nutrients, sun drying is a favorable drying method because forced drying tends to remove vitamin D while sun drying does not.

The wood ear mushrooms that have been dried as described above are then crushed into particulates by a crusher like a rolling mill, a fast-spinning cutter, or a jet mill. Although the particulates may have any particle diameter as long as the particulates are quickly swollen in a stomach, a particle diameter of 125 to 250 μm may be suitable for the particulate powdered wood ear mushrooms to be mixed with matcha to yield a homogeneous mixture.

These particle diameters can be obtained by using a sieve or the like to sort and size particles of wood ear mushrooms crushed by a crusher, so that particles falling in a certain range of particle diameters are prepared by using 80 to 85 mesh as a guide. As a result, there can be provided a stable mixture of the particles and matcha, delivering stabilized quality of the particulate health food.

The matcha used for the present invention is produced by selecting, as a starting material, the tea leaves that are soft and relatively young, such as the first or second picked tea leaves, drying the leaves almost without rolling in the normal green tea production process, and then crushing the leaves with a stone mill or any known mill or sorting the leaves by size. In particular, for efficient mixture with powdered wood ear mushrooms, it is preferable to use the matcha whose particle-size distribution shows a peak at 3 to 10 μm corresponding to a finely crushed thin portion of raw green tea leaves and a peak at 20 to 40 μm corresponding to a crushed thick or vein portion of raw green tea leaves.

As a reference, a human tongue physically feels “granular” particles having a diameter of 30 μm or more, and thus good-quality matcha for drinking has a standard particle diameter of 10 to 20 μm on average. In general, matcha for drinking produced by crushing tea leaves with a stone mill, a ball mill, a hammer mill, or the like has particle sizes distributed in a range from 1 to 100 μm. The present invention can provide desirable results by using such matcha having a particle diameter of 1 to 100 μm with the particle-size distribution showing peaks at 3 to 10 μm and at 20 to 40 μm.

To provide a hard-to-separate and homogenously mixed powder, it is preferable to contain 15 to 35 parts by mass of the above-described matcha having a particle diameter of 1 to 100 μm with respect to 100 parts by mass of the above-described particulate powdered wood ear mushrooms having a particle diameter of 125 to 250 μm.

It is not preferable to contain matcha in an amount as small as less than 15 parts by mass because such matcha fails to fully cover surfaces of a powdered wood ear mushroom, making it more likely that the mixture will separate under vibration. It is not preferable to contain matcha in an amount as large as more than 35 parts by mass because abundant powdered matcha is present more than necessary for covering a powdered wood ear mushroom, which also makes it more likely that the mixture will separate under vibration.

It is preferable to contain 20 to 30 parts by mass, more preferably 23 to 27 parts by mass, of matcha with respect to 100 parts by mass of powdered wood ear mushrooms, because such matcha has the above-described tendencies, is brighter than wood ear mushrooms in color and efficient as an additive, and is capable of coloring the mushrooms well enough to make the mushrooms more palatable.

In order that the aforementioned particulate health food has such matcha content and has better hard-to-separate features against vibrations while the matcha stably covers powdered wood ear mushrooms, it is more preferable that the particulate health food is a granular particulate composition.

The particulate composition or the granular particulate health food can be eaten as a health food by scooping the food with a tool like a spoon to swallow with water or crunch it as appropriate.

The capsuled particulate health food, which is produced by containing the above-described particulate health food in a capsule ingestible with a liquid, can also be ingested or otherwise eaten with another food or drink.

The capsule ingestible with a liquid for the present invention need only be water soluble to the extent that the capsule is dissolved or disintegrated in a human stomach and be made of materials non-hazardous to human bodies. As long as these conditions are satisfied, no specific limitation is imposed on materials, shapes (sphere, oval sphere, cylinder, and so on), and dimensions of the capsule.

For example, a plant-based hard capsule is preferably used as the capsule ingestible with a liquid for the present invention because of its properties of being hard to react with vitamin C contained in matcha, compared with gelatin capsules. Specific examples that may be used include an HPMC capsule (Quali-V-N Capsule supplied by Qualicaps), which is made of hydroxymethylpropyl cellulose derived from plant fibers and is made commercially available as a capsule to contain a health food. Such plant-based hard capsule used as a capsule ingestible with a liquid may be turned into a soft capsule by adding glycerin, sorbitol, or the like to increase the plasticity of the capsule.

The health food that is loaded into such capsule and contains the above-described powdered dry wood ear mushrooms or granular particulate composition may be ingested by swallowing it with water before meal, where suitable quantities of the health food may be determined taking into consideration the age, body weight, or dietary habit of the person who eats the health food.

When taken in this way, the capsule is dissolved and disintegrated in the stomach to quickly expose the contents, that is, low-calorie wood ear mushrooms and matcha, which then absorb water, and the powdered dry wood ear mushrooms expand to eight to ten times the initial volume. Thus, this health food brings a sense of satiety to help reduce or limit caloric intake from food.

A wood ear mushroom (in particular, a black or white one) contains, along with calcium, vitamin D in an amount as immense as 16,000 IU (where 1 IU is equal to 0.026 micrograms). Thus, taking a wood ear mushroom alone enables efficient absorption of calcium into a human body.

Vitamin D is a component involved in bone formation, performing the roles of promoting calcium absorption in the intestines and regulating the amount of calcium releasing from bone. Thus, ingesting wood ear mushrooms to supplement Vitamin D helps prevention of osteoporosis. Furthermore, compelling theories developed by investigators of hormones involved in vitamin D metabolism suggest that ingesting a suitable amount of wood ear mushroom helps prevention of diabetes and adjustment of hormone balance.

Matcha contains a large amount of polyphenols, vitamin C, catechin, caffeine, dietary fibers, amino acids, and minerals, representing a health food with recognized effects of antioxidation and recovery from fatigue. Ingesting a wood ear mushroom in combination with such matcha is expected to improve recovery from fatigue and immune functions. Therefore, the health food serves not only for dieting purposes but also for health of aged people.

### EXAMPLES

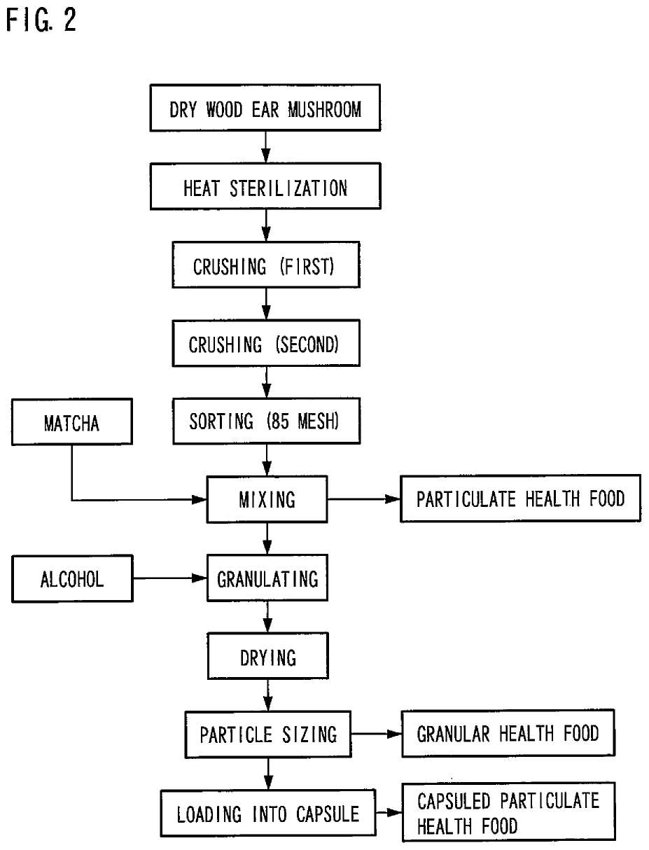

In accordance with the production process shown in the flowchart in FIG. 2, the main ingredient, powdered wood ear mushrooms, was prepared by: crushing (first step) dried wood ear mushrooms that had already been thoroughly washed with drinking water into particles of about 1 mm in average diameter through a roll crusher; further crushing (second step) the particles into about 0.2 mm-size particles followed by heat sterilization; and sorting and sizing the particles through 85 mesh (sieve opening: about 170 μm).

Regarding the other material, matcha, commercially available drinking matcha was used. The matcha had a particle diameter of 1 to 100 μm, 5.8 μm on average, with the particle-size distribution showing peaks at about 4 μm and 22 μm, which were identified through measurement by a dry method with a laser diffraction particle size distribution measuring device.

Next, the materials, specifically 100 parts by mass of the particulate powdered wood ear mushrooms and 25 parts by mass of the drinking matcha, were homogeneously mixed by a mixer, thereby producing 1 kg of a particulate health food.

On the obtained particulate health food, the below-mentioned vibration test was conducted in order to find out whether the mixture of the powdered wood ear mushroom and the powdered coloring component was not separated by a vibration caused before the mixture was loaded into a capsule or the like.

Specifically, about 10 ml of the particulate health food was contained in a test tube, and a handheld small vibration tester was used to vibrate the whole test tube horizontally five times per second with an amplitude of 5 mm for two minutes, and then layers formed in the test tube, if any, were observed.

It was found that the composition remained a homogeneous mixture, without separating into layers by particle diameter of the powdered particles.

Next, the particulate health food was granulated with an aqueous alcohol by a fluid bed granulator, further subjected to hot drying, and sized into about 2 mm granules in average particle diameter of the particulate health food, which were then loaded into an HPMC capsule having a capacity of 350 mg, which is a commercially available plant-based hard capsule. In this way, a capsuled particulate health food was produced.

Eight to ten capsules of the obtained capsuled particulate health food were ingested with 200 cc of water 30 minutes before meal by each of five adult males and five adult females, and every panelist gave his/her impression that the food brought a moderate sense of satiety. Thus, the food was found to be promising as a low-calorie health food providing dietary effects.

### Comparative Example

A particulate mixture was produced in the same way as in Examples except that flour having an average particle diameter of 25 μm was used instead of matcha.

A vibration test was conducted on the obtained particulate mixture under exactly the same conditions as those described above, resulting in a heterogeneous mixture having approximately separate layers of flour and powdered wood ear mushrooms under vibration.

### REFERENCE SIGNS LIST

1 Powdered wood ear mushroom
2a Large matcha particle
2b Small matcha particle
2 Matcha particles

1. A particulate health food comprising a particulate composition that includes wood ear mushrooms as a main component, the wood ear mushrooms comprising powdered particulate dry wood ear mushrooms having a particle diameter of 125 to 250 μm, and wherein the particulate composition further includes, based on 100 parts by mass of the wood ear mushrooms, 15 to 35 parts by mass of matcha having a particle diameter of 1 to 100 μm.

2. The particulate health food according to claim 1, wherein the matcha has a particle-size distribution in which peaks appear at 3 to 10 μm and 20 to 40 μm.

3. The particulate health food according to claim 1, wherein the particulate health food is a granular particulate composition.

4. A capsuled particulate health food, wherein the particulate health food according to claim 1 is contained in a capsule ingestible with a liquid.

5. The particulate health food according to claim 2, wherein the particulate health food is a granular particulate composition.

6. A capsuled particulate health food, wherein the particulate health food according to claim 2 is contained in a capsule ingestible with a liquid.

7. A capsuled particulate health food, wherein the particulate health food according to claim 3 is contained in a capsule ingestible with a liquid.

8. A capsuled particulate health food, wherein the particulate health food according to claim 5 is contained in a capsule ingestible with a liquid.
